(19) United States
(12) Patent Application Publication
(10) Pub. No.: US 2009/0018452 A1
(43) Pub. Date: Jan. 15, 2009

(54) PROBE ADAPTED TO MEASURE BIOLOGICAL SIGNAL

(75) Inventors: Keiichi SUGIURA, Tokyo (JP); Toru MAEDA, Tokyo (JP); Teiji UKAWA, Tokyo (JP); Shinji YAMAMORI, Tokyo (JP)

Correspondence Address: SUGHRUE-265550, 2100 PENNSYLVANIA AVE. NW, WASHINGTON, DC, 20037-3213, US

(73) Assignee: NIHON KOHDEN CORPORATION, Tokyo (JP)

(21) Appl. No.: 12/059,719

(22) Filed: Mar. 31, 2008

(30) Foreign Application Priority Data
Mar. 30, 2007 (JP) P.2007-094765
Feb. 14, 2008 (JP) P2008-032926
Mar. 28, 2008 (JP) P2008-87520

(51) Int. Cl. A61B 6/00

(52) U.S. Cl. 600/476

(57) ABSTRACT

A probe is adapted to emit light having at least one wavelength as irradiation light with respect to a measured portion on a biological body in order to measure a biological signal. A light emitting element is configured to emit the light having the at least one wavelength. A light guiding member is provided with: a first end face, being light-reflective and having a first part opposing the light emitting element and a second part surrounding the first part; a second end face, being light-reflective and intersecting the first end face; and a third end face, being light-permeable, opposing the first end face and intersecting the second end face. The light guiding member is configured such that at least part of the light emitted from the light emitting element is reflected by at least one of the first end face and the second end face, and emitted from the third end face as the irradiation light. A light receiving element is configured to receive light reflected by the measured portion. A section shape of the first end face in a direction along an optical axis of the light emitting element is such a shape that at least the second part is coincident with a circumference of an ellipse intersecting a minor axis of the ellipse, and the optical axis of the light emitting element is coincident with the minor axis of the ellipse.

### BACKGROUND

The present invention relates to a probe for accurately measuring a biological signal.

A pulse monitor which is a kind of the probe for measuring the biological signal is suggested in the past (see Japanese Patent Publication No. 2000-116611A; hereinafter referred to as Patent Document 1).

In the pulse monitor described in the above document, light emitted from a light emitting diode is reflected by a convex mirror and the light reflected and spherically-spread is further reflected by a concave mirror toward a light emitting face. A space between the concave mirror and the light radiating surface is filled with a light diffusing agent to diffuse the light to be radiated. It is disclosed that, by diffusing the light emitted from the light source in this manner, a light plane in which a light intensity is made uniform can be emitted from the light emitting face to irradiate a measured object. In the above configurations, the light emitting diode is disposed in the vicinity of a measured part such as a finger and a foot, when the measurement is performed.

As the same type of the probe for measuring the biological signal, it is well-known a configuration in which an LED serving as a light emitting element and a PD serving as a light receiving element are arranged within the same plane and covered with a lens which is convex toward a measured object (see Japanese Patent Publication No. 2005-505360T; hereinafter referred to as Patent Document 2).

Further, it is well-known a device for measuring reflected light in which a light emitting diode (LED) and a light receiving element (PD) are arranged in the vicinity of a surface of a measured object in a vertical direction relative to the surface of the measured object (see Japanese Patent Publication No. 58-33153A; hereinafter referred to as Patent Document 3).

In the reflection-type probe for measuring the biological signal described in Patent Document 3, since the light emitting element is disposed on a line connecting the light receiving element and the measured object, a tubular light guiding member surrounding the light emitting element is used to guide the light reflected by the measured object. The light guiding member is transparent or translucent and side end faces thereof are made reflective. When the measured object is irradiated with the light emitted from the light emitting element, the light reflected by the measured object enters the light guiding member, travels in the light guiding member while being reflected by the side end faces, and is finally incident on the light receiving element located in the back of the light emitting element as viewed from the measured object.

According to this configuration, the width can be reduced in comparison with the configuration in which the light emitting element and the light receiving element are laterally arranged as in the probe described in Patent Document 2. However, since the tubular light guiding member should extend in the thickness direction of the probe, the thickness dimension is increased.

On the other hand, it is well-known a reflection-type probe for measuring a biological signal in which a light emitting element is disposed above a light receiving element so that a measured portion of a biological body is irradiated with light emitted from the light emitting element and passed through a light guiding member, thereby causing light reflected by the measured portion to be directly incident on the light receiving element. In this probe, the light guiding member covers an upper part of the light emitting element and a face opposing the light emitting element is made reflective. In order to efficiently irradiate the measured portion, the light reflecting face is made concave and semi-spherical. Accordingly, the light guiding member should have a semi-spherical shape, which causes the increase in size in the thickness direction of the probe.

### SUMMARY

It is therefore one advantageous aspect of the invention to provide a probe for measuring a biological signal that is reduced in size in the thickness direction thereof, and that can irradiate a biological body with light emitted from a light emitting element with loss suppressed as much as possible, by improving the configuration of a light guiding member guiding the light from the light emitting element to the biological body.

According to one aspect of the invention, there is provided a probe, adapted to emit light having at least one wavelength as irradiation light with respect to a measured portion on a biological body in order to measure a biological signal, the probe comprising:

a light emitting element, configured to emit the light having the at least one wavelength;

a light guiding member, comprising:

a first end face, being light-reflective and having a first part opposing the light emitting element and a second part surrounding the first part;

a second end face, being light-reflective and intersecting the first end face; and

a third end face, being light-permeable, opposing the first end face and intersecting the second end face,

the light guiding member configured such that at least part of the light emitted from the light emitting element is reflected by at least one of the first end face and the second end face, and emitted from the third end face as the irradiation light; and

a light receiving element, configured to receive light reflected by the measured portion, wherein:

a cross-sectional shape of the first end face in a direction along an optical axis of the light emitting element is such a shape that at least the second part is coincident with a circumference of an ellipse intersecting a minor axis of the ellipse, and the optical axis of the light emitting element is coincident with the minor axis of the ellipse.

The probe may be configured such that: the light emitting element is disposed at a position corresponding one of focuses of the ellipse; at least the second part of the first end face is concave with respect to the light emitting element; and a point on the second end face is located at a position corresponding to the other one of the focuses of the ellipse.

The probe may be configured such that: the light emitting element is disposed at a position corresponding one of focuses of the ellipse; the first part of the first end face is convex with respect to the light emitting element as a first convex part; and the second part of the first end face is concave with respect to the light emitting element.

The first convex part may be semi-spherical or conical.

A second convex part which is semi-spherical or conical and convex with respect to the light emitting element may be formed on the first convex part.

The may further comprise: a support, on which the light emitting element is mounted; and a planar mirror provided on a part of the support opposing the first end face. Here, the light emitted from the light emitting element is reflected by the first end face and the planar mirror to be guided to the third end face.

The light emitted from the light emitting element may be reflected by the first part of the first end face and the second end face to be guided to the third end face.

The probe may further comprise a support, on which the light emitting element is mounted. Here, a part of the support opposing the first end face is convex with respect to the first end face and light-reflective.

The first part and the second part of the first end face may be convex with respect to the light emitting element.

The probe may further comprise a wire, electrically connected to the light emitting element and the light receiving element. Here, the wire is led out from the second end face.

The probe may further comprise a board, having a first face including a first region and a second region, the board being a folded state that the first region and the second region are directed to opposite directions. Here, the light emitting element is disposed on the first region and the light receiving element is disposed on the second region.

The probe may further comprise a layer disposed between the first region and the second region, and having a thermal conductivity which is lower than a thermal conductivity of the board.

According to the biological signal measuring probe of the invention, it is possible to reduce the thickness and size of the probe by improving the configuration of the light guiding member for irradiating the biological body with light emitted from the light emitting element. In addition, it is possible to reduce the loss of light emitted from the light emitting element and incident on the biological body.

### BRIEF DESCRIPTION OF THE DRAWINGS

FIG. 1 is a cross-sectional view of a probe for measuring a biological signal according to a first embodiment of the invention.

FIG. 2 is a cross-sectional view of a probe for measuring a biological signal according to a second embodiment of the invention.

FIG. 3 is a cross-sectional view of a probe for measuring a biological signal according to a third embodiment of the invention.

FIGS. 4A and 4B are views showing wirings to a light emitting element and a light receiving element in the probe of the invention.

FIGS. 5A and 5B are views for explaining how to arrange the light emitting element and the light receiving element on a board, and how to mount the board on the probe of the invention.

FIG. 6A is a cross-sectional view of a probe for measuring a biological signal according to a fourth embodiment of the invention.

FIG. 6B is a cross-sectional view of a probe for measuring a biological signal according to a fifth embodiment of the invention.

FIG. 6C is a cross-sectional view of a probe for measuring a biological signal according to a sixth embodiment of the invention.

FIGS. 7A to 7C are views showing difference in a reflected state of light emitted from an LED element in accordance with difference of a shape of a light guiding member.

FIGS. 8A and 8B are views showing evaluation results of the reflected state of the light emitted from the LED element in accordance with the shape of the light guiding member.

FIG. 9A to 9C are views showing difference in a reflected state of light emitted from an LED element in accordance with a distance between the LED element and a lower end face of the light guiding member, and with a shape of a face on which the LED element is mounted.

FIGS. 10A and 10B are views showing evaluation results of the reflected state of the light emitted from the LED element in accordance with the distance between the LED element and the lower end face of the light guiding member, and with the shape of the face on which the LED element is mounted.

FIGS. 11A and 11B are views showing a probe for measuring a biological signal according to an eighth embodiment of the invention.

FIGS. 12A to 12D are view showing how are red light (R) and infrared light (IR) spread when the LED element directly irradiate skin of a patient.

FIGS. 13A and 13B are views showing a probe for measuring a biological signal according to a ninth embodiment of the invention.

FIGS. 14A and 14B are views showing a probe for measuring a biological signal according to a tenth embodiment of the invention.

FIG. 15 is a section view showing a probe for measuring a biological signal according to an eleventh embodiment of the invention.

FIGS. 16A and 16B are views showing fluctuations of an intensity waveform of transmitted light and variations of amplitude of pulse wave when the LED element irradiates a measured object with light having a single wavelength.

### DETAILED DESCRIPTION OF EXEMPLIFIED EMBODIMENTS

Exemplified embodiments of the invention will be described below in detail with reference to the accompanying drawings. Among the different embodiments, common or similar components will be designated by the same reference numerals and repetitive explanations thereof will be omitted.

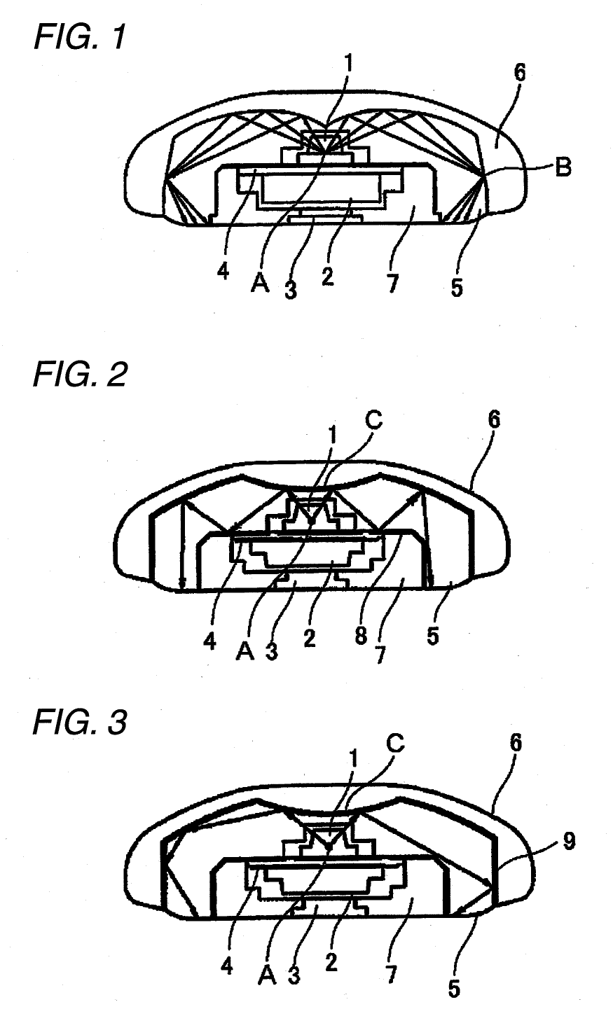

FIG. 1 shows a probe for measuring a biological signal according to a first embodiment of the invention. An LED element 1 serving as a light emitting element is mounted on an upper face of a printed circuit board 4. At least one LED chip is disposed in the LED element 1. For example, two or more LED chips for emitting light having different wavelengths (e.g., red light around 660 nm infrared light around 900 nm may be disposed.

A PD element 2 serving as a light receiving element is mounted on a lower face of the printed circuit board 4 so as to oppose a light receiving window 3.

A light guiding member 5 is disposed so as to surround the LED element 1 and the PD element 2. The light guiding member 5 is formed of a transparent molded material having high transparency and high optical characteristic such as polyethylene (PET), polycarbonate, and acryl. A light diffusing material having a milky white color such as titanium oxide filler may be mixed therewith. The end face of the light guiding member 5 serves as a light reflecting face.

The light emitted from the LED element 1 travels in the light guiding member 5, is reflected by the light reflecting face, and is guided to a lower end face of the light guiding member 5, which serves as a light emitting face of the probe. When the light emitted from the light emitting face of the probe is incident on a measured object, the light reflected by the measured object is incident on the PD element 2 through the light receiving window 3.

A light-shielding cover 6 and a light-shielding case 7 are disposed above and below the light guiding member 5, respectively. The light-shielding cover 6 serves not to mix ambient light into the measuring irradiation light (the light emitted from the LED element 1) and the light-shielding case 7 serves to prevent the irradiation light from the light guiding member 5 from entering the light receiving window 3 without being reflected by the biological body.

The light reflecting face may be constructed by covering the outer end face of the light guiding member 5 with a metal coating such as an aluminum foil or by forming the inner face of the light-shielding cover 6 or the light-shielding case 7 as a high-reflectance surface.

The light-shielding cover 6 above the light guiding member may be omitted and a member used to attach the probe on a measured portion of a patient may be also used as the light-shielding cover.

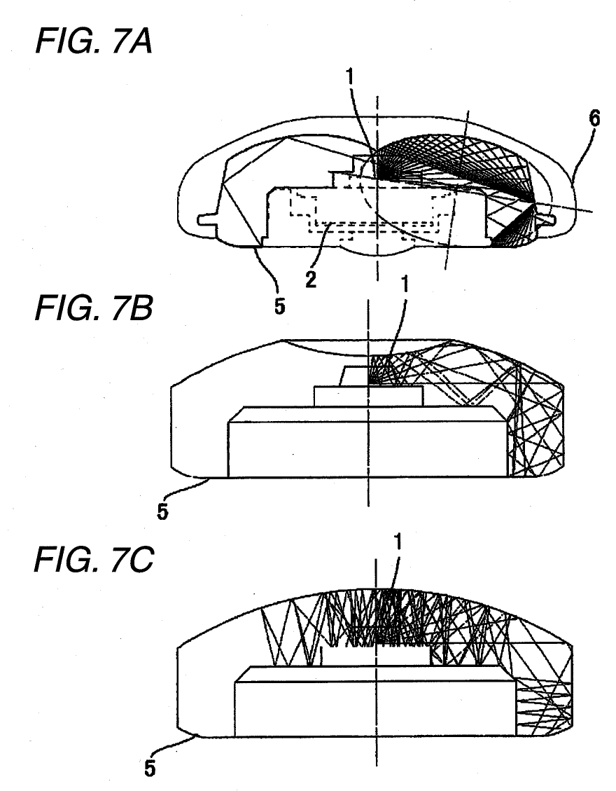

As shown in FIG. 1, the upper end face of the light guiding member 5 has a laterally-elongated arc-shaped cross section along an optical axis of the LED element 1. The upper end face is formed as a concave mirror as viewed from the LED element 1. More specifically, as shown in FIG. 7, the cross section has a shape coincident with a part of the circumference of an ellipse intersecting a minor axis of thereof. The light source of the LED element 1 is disposed at a position coincident with a focus A of the ellipse. One point on the side end face of the light guiding member 5 is made coincident with a focus B of the ellipse.

In other words, the upper end face of the light guiding member 5 constitutes a curved face as a set of points where the sum of the distance from a light source position A of the LED element 1 and a distance from a point B in the side end face is constant. The curved face includes the point where the distance from the light source position A of the LED element 1 is equal to the distance from the point B in the side end face (that is, the minor axis of the ellipse).

The upper end face of the light guiding member 5 may be considered as a curved face formed by a part of the circumference intersecting the minor axis of the ellipse when the ellipse is rotated about the focus A around the optical axes of the LED element 1 and the PD element 2.

As shown in FIGS. 1 and 7, since the LED element 1 is disposed at the focus A of the ellipse, the light having at least one wavelength emitted from the LED element 1 is reflected by the upper end face (concave mirror) of the light guiding member 5 having an arc shape corresponding to the circumference of the ellipse and is concentrated on the side end face of the light guiding member 5 corresponding to the other focus B. The light reflected by the side end face is guided to the bottom face of the light guiding member 5 and is emitted as light for irradiating the measured object from the lower portion of the probe (the lower end face of the light guiding member 5).

According to the configuration of this embodiment, it is possible to reduce the size in the thickness direction in comparison with the light guiding member having the known semi-spherical light reflecting face, thereby contributing to the decrease in size of the probe. On the other hand, since the light emitted from the LED element 1 can be efficiently guided to the lower end face of the light guiding member 5, it is possible to emit the irradiating light from the probe with loss suppressed as much as possible. Since the light diffusing material is contained in the light guiding member 5, the light intensity emitted from the probe is made uniform regardless of the position on the lower end face of the light guiding member 5, thereby enabling the accurate measurement.

A biological signal measuring probe according to a second embodiment of the invention is shown in FIG. 2. The second embodiment is different from the first embodiment shown in FIG. 1 in the following points.
A portion C of the upper end face of the light guiding member 5 opposing the LED element 1 forms a semi-spherical portion convex toward the LED element 1 so as to serve as a convex mirror.
A surface 8 of the light-shielding case 7 opposing the convex mirror is formed in a plane mirror.

According to the configuration shown in FIG. 2, the light emitted from the LED element 1 is first reflected by the convex mirror C and is then reflected by the plane mirror 8. Thereafter, the light reflected by the upper end face of the light guiding member 5 is emitted as the light irradiating the measured object from the lower portion of the probe (the lower end face of the light guiding member 5).

According to the configuration shown in FIG. 2, since the ceiling portion of the light guiding member 5 can be formed in a concave shape, it is possible to reduce the thickness (height) of the light guiding member and thus to reduce the entire thickness of the probe (a part of the probe to be attached on the measured object). Since the light reflected by the upper end face and the plane mirror 8 is guided to the lower end face without passing through the side end face, the side end face may not be made coincident with the focus B of the ellipse, unlike the first embodiment. That is, the position of the side end face may be shifted further inward in comparison with the first embodiment. Accordingly, it is possible to reduce the lateral width of the probe.

FIG. 3 shows a biological signal measuring probe according to a third embodiment of the invention.

The third embodiment is different from the first embodiment shown in FIG. 1 in the following points.
A portion C of the upper end face of the light guiding member 5 opposing the LED element 1 forms a semi-spherical portion convex toward the LED element 1 so as to serve as a convex mirror.
The side end face of the light guiding member 5 is located outside the focus B of the ellipse and is formed in a reflecting face 9. The reflecting face 9 may be a plane or a curved face.

According to the configuration shown in FIG. 3, the light emitted from the LED element 1 is first reflected by the convex mirror C, is then reflected by the reflecting face 9 of the side end face of the light guiding member 5, and is finally emitted as the light irradiating the measured object from the lower portion of the probe (the lower end face of the light guiding member 5).

According to the configuration shown in FIG. 3, since the ceiling portion of the light guiding member 5 can be formed in a concave shape, it is possible to reduce the thickness (height) of the light guiding member. Since the plane mirror 8 may not be formed in the light-shielding case 7 unlike the second embodiment shown in FIG. 2, it is possible to reduce the manufacturing cost. Since the number of reflection is small, it is possible to suppress the loss of the light emitted from the LED element 1 until the light is output from the probe.

Next, a method of allowing a wire to extend from the light emitting element and the light receiving element to the outside of the biological signal measuring probe will be described now. This explanation can be applied to all the above embodiments and all embodiments described later.

In the conventional biological signal measuring probe, the wire extending from the light emitting element and the light receiving element to the outside of the probe is generally drawn out along a bottom face (a contact face with a biological body) of the probe.

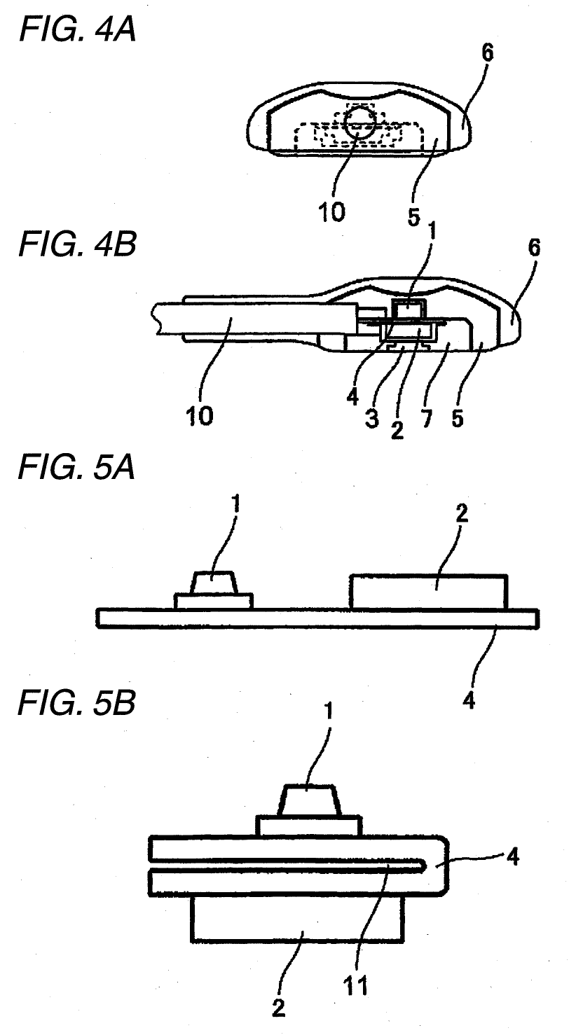

In the biological signal measuring probe according to the invention, the wire extending from the light emitting element and the light receiving element to the outside of the probe has the configuration shown in FIGS. 4A and 4B.

Specifically, a wire 10 is introduced from a side of the probe, is connected to the LED element 1 serving as the light emitting element and the PD element 2 serving as the light receiving element, and is drawn outward from the probe.

With this configuration, since the light beam incident on the biological body from the bottom of the probe is not blocked by the wire, it is possible to efficiently irradiate the biological body with the light beam.

Next, arrangement of the light emitting element and the light receiving element on the board and attachment of the board to the biological signal measuring probe according to the invention will be described. This explanation can be applied with respect to all the above embodiments and all embodiments described later (except an eleventh embodiment).

As shown in FIG. 5A, the LED element 1 as the light emitting element and the PD element 2 as the light receiving element are arranged on the same face of the board 4. Since components (the LED and the PD) can be mounted on only one side of the board, it is possible to simplify the manufacturing process.

A flexible printed circuit board or the like being easily subjected to a fold-back process is used as the board. At the time of arranging the board in the probe, the board 4 is folded back as shown in FIG. 5B, so that the LED element 1 is disposed on the top face of the board and the PD is disposed on the bottom face.

By interposing a material having low thermal conductivity into a gap 11 formed between the folded-back board, the heat generated from the LED element is hardly transmitted to the biological body through the board 4. The material having low thermal conductivity includes air.

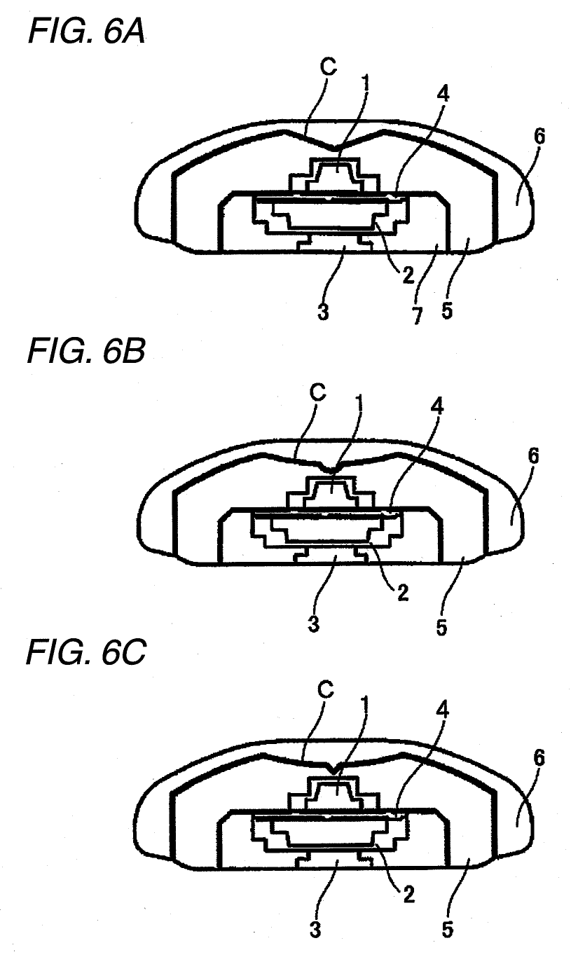

FIG. 6A shows a biological signal measuring probe according to a fourth embodiment of the invention. The fourth embodiment is equivalent to the second embodiment shown in FIG. 2, except that the convex portion C formed on the upper end face of the light guiding member 5 is formed in a conical shape and is formed in a conical convex mirror. This configuration can be applied to the third embodiment shown in FIG. 3.

FIG. 6B shows a biological signal measuring probe according to a fifth embodiment of the invention. The fifth embodiment is equivalent to the second embodiment shown in FIG. 2, except that the convex portion C formed on the upper end face of the light guiding member 5 includes two large and small semi-spherical surfaces and each of them is formed in a semi-spherical convex mirror. This configuration can be applied to the third embodiment shown in FIG. 3.

FIG. 6C shows a biological signal measuring probe according to a sixth embodiment of the invention. The sixth embodiment is equivalent to the second embodiment shown in FIG. 2, except that the convex portion C formed on the upper end face of the light guiding member 5 includes a semi-spherical portion and a conical portion formed at the end thereof and the semi-spherical portion and the conical portion is formed in a semi-spherical convex mirror and a conical convex mirror, respectively. This configuration can be applied to the third embodiment shown in FIG. 3.

Next, a difference in output light intensity with respect to a measured object due to a difference in shape of the light guiding member will be described. The estimation is carried out as explained below.

The output light intensity (at the light emitting face) from the probe was obtained by analyzing a light beam by a step of 10 degree from 0 degree to 80 degree about the optical axis of the LED element 1. When it is assumed that the directional characteristic of the LED element 1 does not vary with an angle, the output light intensity (LED output light intensity) at the entire angles is 1, the output light intensity from the probe was calculated by multiplying the output light intensity by a value obtained by raising the attenuation rate 0.97 due to the beam reflection to the N-th power only in consideration of the attenuation due to the reflection. Here, N represents the number of reflecting times. That is, the output light intensity is expressed as a percentage with the output light intensity of the LED element 1, which is hereinafter referred to as “output light intensity ratio”.

FIGS. 7A to 7C show the difference of reflection acting on the light beam emitted from the LED element 1 due to the difference in shape of the light guiding member 5. FIG. 7A shows the first embodiment shown in FIG. 1 (sample I). FIG. 7B shows the second embodiment shown in FIG. 2 (sample II). FIG. 7C shows a sample in which the upper end face of the light guiding member 5 does not have a convex portion protruding toward the LED element 1 just above the LED element 1 (sample III). In this embodiment, the sectional shape of the upper end face of the light guiding member 5 along the optical axis of the LED element 1 corresponds to a part of the circumference intersecting the minor axis of the ellipse so that the optical axis of the LED element 1 is coincident with the minor axis of the ellipse.

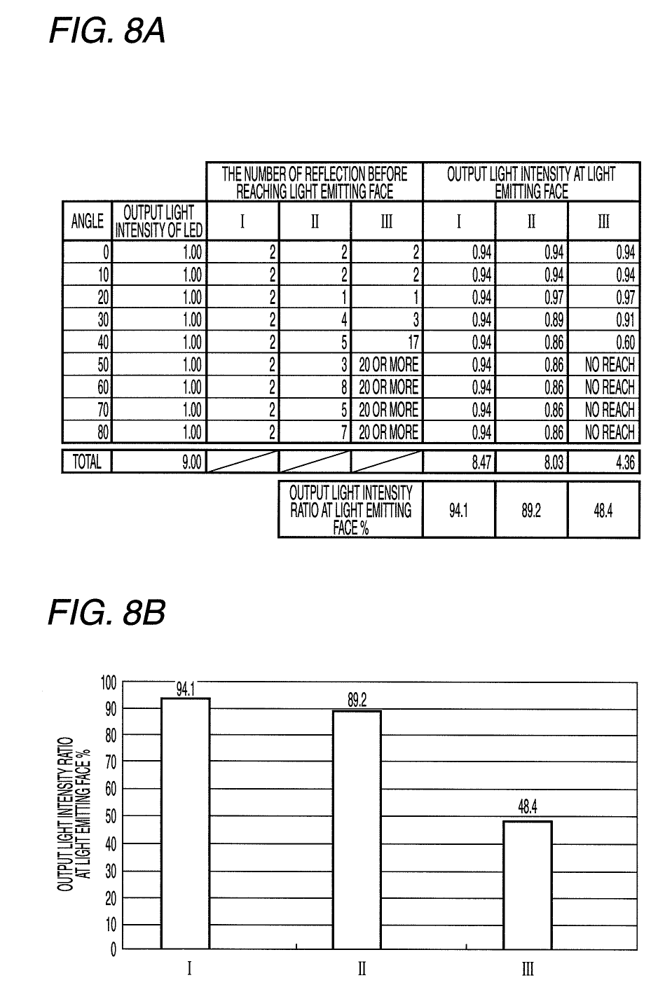

The estimation result of the output light intensity from the probe due to the difference in shape of the light guiding member 5 is shown in FIGS. 8A and 8B. FIG. 8A is a table illustrating the result of the light beam analysis and FIG. 8B is a graph expressing the result.

It can be seen from the graph shown in FIG. 8B that the output light intensity ratio of sample I is 94.1% which is the highest and the loss is very small. It can be also seen that the output light intensity ratio of sample II is 89.2% which is satisfactory. In comparison with sample III, it can be seen that the shape design of the upper end face of the light guiding member 5 greatly contributes to the improvement in output light intensity.

The difference in reflection of the light beam emitted from the LED element 1, due to the height (thickness) of the light guiding member 5 in the biological signal measuring probe and the difference in shape of the surface (LED mounting face) of the light-shielding case 7 opposing the upper end face of the light guiding member 5, will be described with reference to sample III.

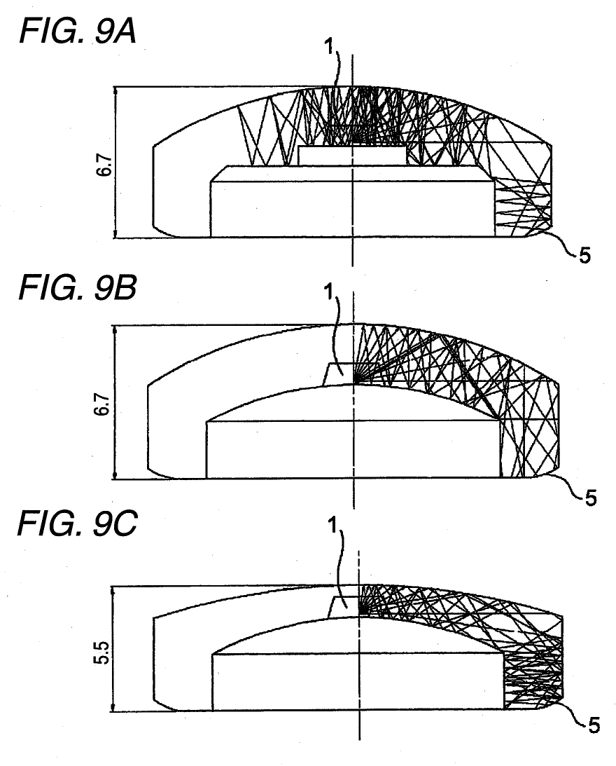

FIG. 9A shows an example corresponding to sample III shown in FIG. 7C in which the height from the lower end face of the light guiding member 5 is 6.7 mm and the LED mounting face of the light-shielding case 7 is formed in a reflecting plane.

FIG. 9B shows an example in which the height from the lower end face of the light guiding member 5 is 6.7 mm and the LED mounting face of the light-shielding case 7 is formed in a semi-spherical reflecting face (sample IV).

FIG. 9C shows an example in which the height from the lower end face of the light guiding member 5 is 5.5 mm and the LED mounting face of the light-shielding case 7 is formed in a semi-spherical reflecting face (sample V).

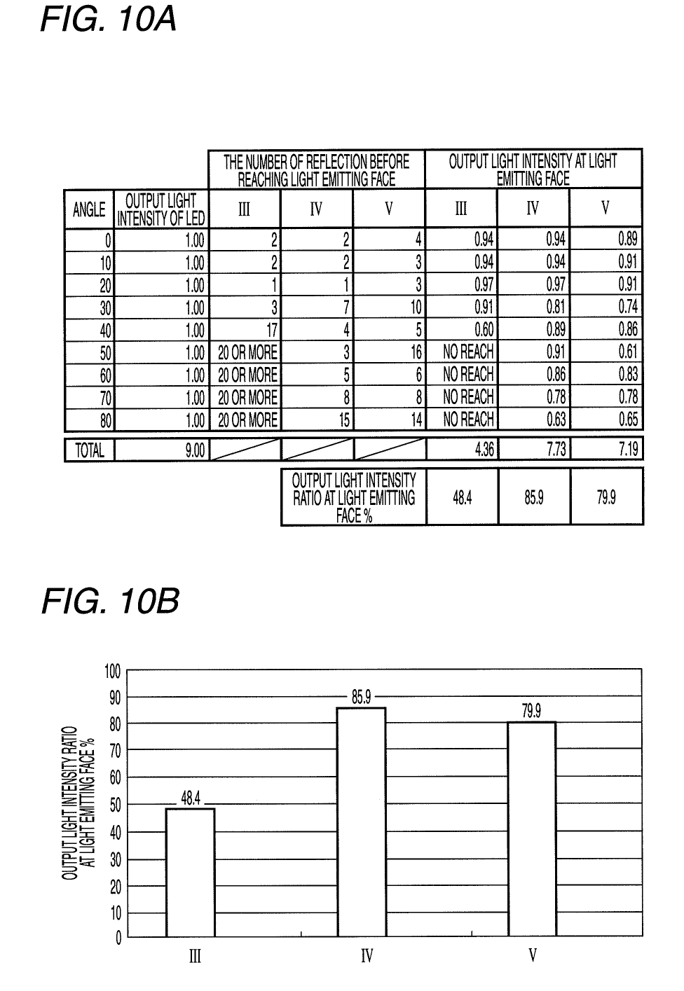

The estimation result of the output light intensity from the probe with the above difference in structure is shown in FIGS. 10A and 10B. The estimation method is the same as performed on samples I to III. FIG. 10A is a table showing the above light beam analysis result and FIG. 10B is a graph illustrating the result thereof.

It can be seen from the graph shown in FIG. 10B that the LED mounting face formed in a curved face is greater in output light intensity than that formed in a plane and the output light intensity increases as the height from the lower end face of the light guiding member 5 increases. This fact teaches that the output light intensity can be enhanced by properly selecting the height and forming the LED mounting face in a curved face even when the light guiding member 5 having the upper end face shape similar to sample III. This configuration is regarded as a seventh embodiment of the invention.

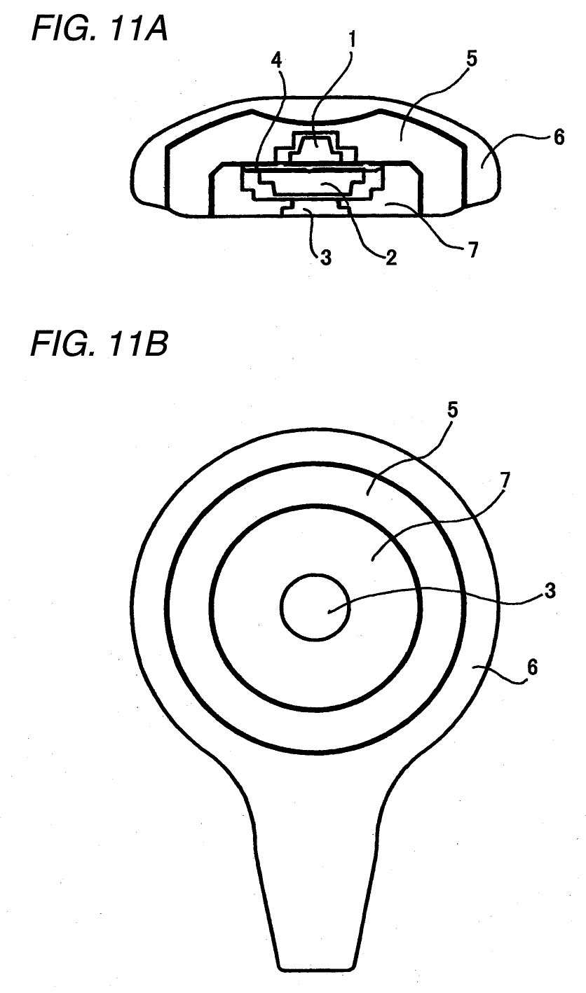

FIGS. 11A and 11B show a biological signal measuring probe according to an eighth embodiment of the invention.

This embodiment is based on the fact that, when a light emitting element (LED) of the conventional biological signal measuring probe which emits heat is disposed in the vicinity of the surface of the measured object (skin of a patient), the skin is affected and burned by the heat of the light emitting element (LED) (see Anaesthesla, 2005, 60, pages 1249-1250 “Reflectance pulse oximeter-associated burn in a critically ill patient”).

This embodiment is so configured as to solve the problem occurred by the configuration in which a plurality of light emitting elements (LED elements) and a light receiving element (PD element) are arranged on the same face such that the PD element is surrounded by the LED elements (the configuration described in Patent Document 1) in a case where the vascular bed in the vicinity of the LED elements is not uniform. According to this embodiment, it is possible to provide a biological signal measuring probe that can be easily mounted on a patient and can make reliable measurement because it is possible to measure a biological signal without causing any low-temperature burn in a measured portion of a patient and to provide an operator with a high degree of freedom for selecting the measured portion.

In this embodiment, a plurality of LED chips for emitting light beams having different wavelengths, for example, red (around 660 nm) and infrared (around 900 nm) are disposed in the LED element 1.

The characteristics of red (around 660 nm) and infrared (around 900 nm) having different wavelengths from two chips mounted into the light emitting element will be reviewed.

The distance between the LED chips is about 1 mm and the beams emitted from the LED chips are diffused more and more as the distance from a light source increases.

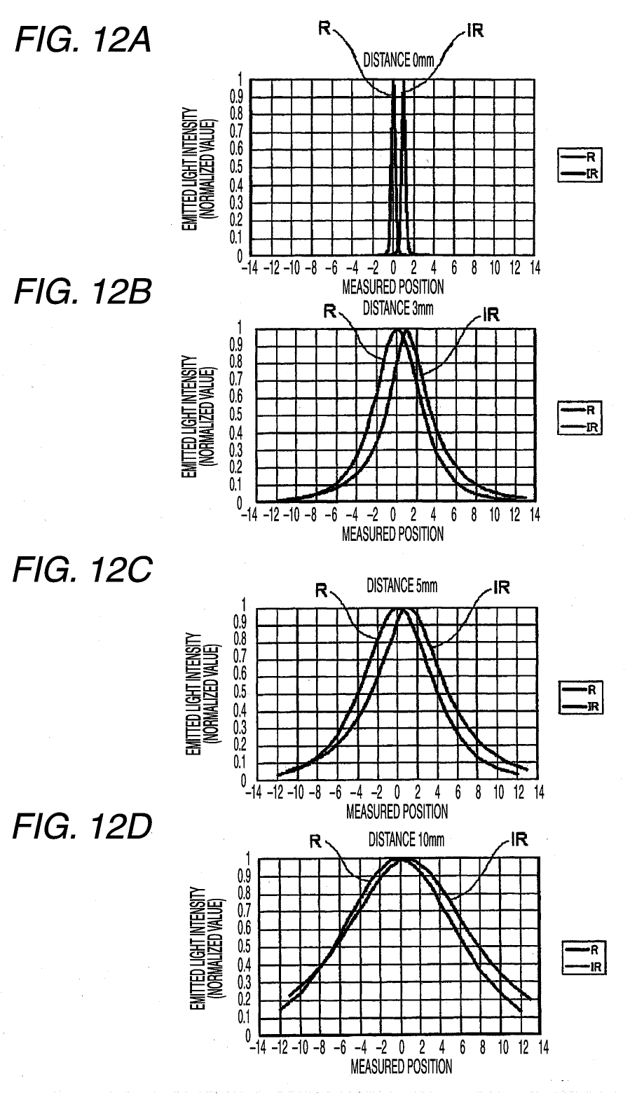

The degree of diffusion of the red beam (R) and the infrared beam (IR) will be described with reference to FIGS. 12A to 12C when a patient's skin is irradiated directly by the LED element.

FIG. 12A shows a case where the patient's skin is located just below the LED element. It can be seen from this figure that the red beam (R) and the infrared beam (IR) are incident on different portions of the patient's skin with hardly overlapping with each other.

FIG. 12B shows a case where a patient's skin is located apart by 3 mm from the LED element. It can be seen from this figure that the red beam (R) and the infrared beam (IR) overlap with each other and are incident on the same portion of the patient's skin.

FIG. 12C shows a case where the patient's skin is located apart by 5 mm from the LED element. It can be seen from this figure that the red beam (R) and the infrared beam (IR) overlap with each other more than in FIG. 12B and are more incident on the same portion of the patient's skin.

FIG. 12D shows a case where the patient's skin is located apart by 10 mm from the LED element. It can be seen from this figure that the red beam (R) and the infrared beam (IR) overlap with each other further more than in FIG. 12C and are further more incident on the same portion of the patient's skin.

In view of the principle of the pulse oximeter on the assumption that the red beam (R) and the infrared beam (IR) are incident on the same portion of the patient's skin and the transmitted or reflected beams are received by the light receiving element, the assumption of the pulse oximeter is not established in FIG. 12A, thereby causing an error.

In FIGS. 12C and 12D, there is a problem that the ratio of applying the beams to different portions of the patient's skin is reduced, but the thickness (height) of the probe increases as the LED element goes apart from the patient's skin.

In the biological signal measuring probe of this embodiment, the beams emitted from the LED element 1 is not applied directly to the patient's skin, but the red beam (R) and the infrared beam (IR) are incident on the same portion of the patient's skin in a state where the distance from the LED element 1 to the patient's skin is increased by causing the beams to travel through the light guiding member 5 and the beams are diffused in the light guiding member 5.

The irradiating beams emitted from the lower end face of the light guiding member 5 are reflected by the measured portion of the biological body and are received by the PD element 2 through the light receiving window 3 while the red beam (around 660 nm) and the infrared beam (around 900 nm) are distinguished from one another.

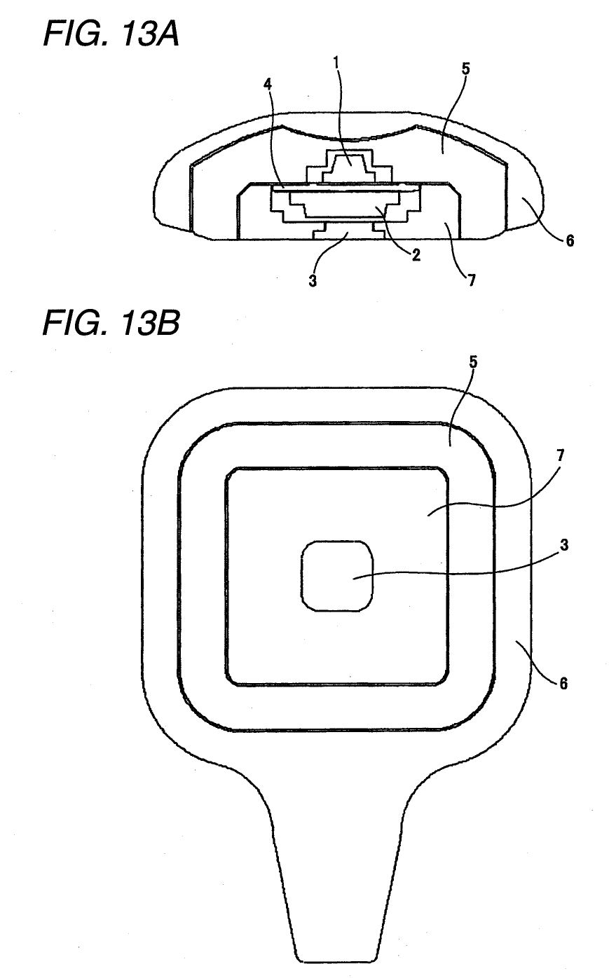

FIGS. 13A and 13B show a biological signal measuring probe according to a ninth embodiment of the invention. This embodiment is different from each of the above embodiments in shape of the probe.

In the above embodiments, the light-shielding case 7, the light guiding member 5, and the light-shielding cover 6 are concentrically disposed about the light receiving window 3 of the circular light receiving element (for example, see FIG. 11B). In this embodiment, as shown in FIG. 13B, the light-shielding case 7, the light guiding member 5, and the light-shielding cover 6 are concentrically disposed about the light receiving window 3 of the substantially rectangular light receiving element.

FIG. 13A is a cross-sectional view taken along a diagonal of the rectangular probe. The sectional shape of the light guiding member 5 in each of the above embodiments may be applied as required.

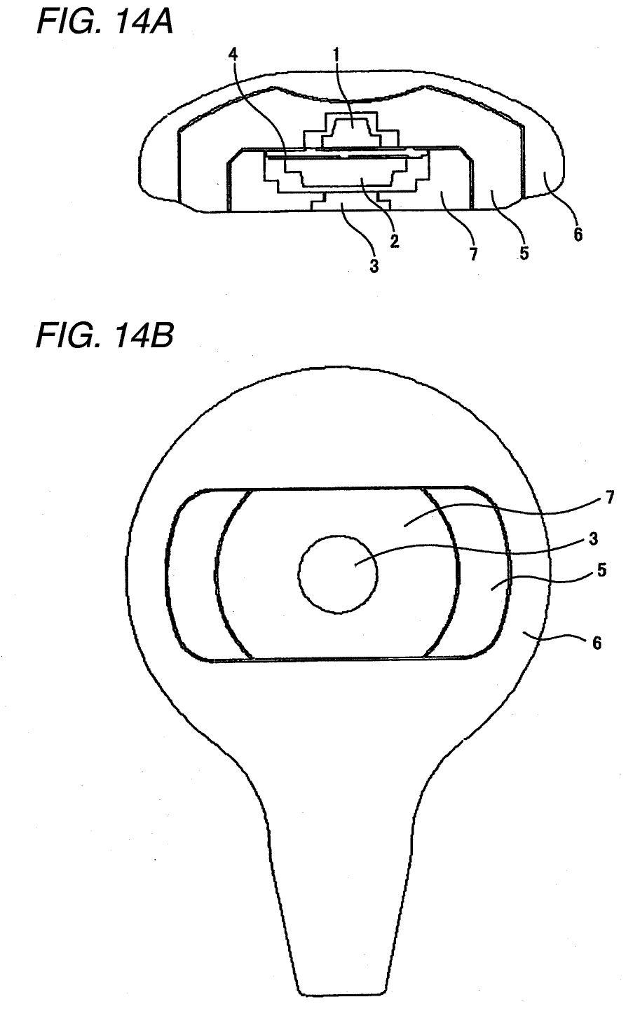

FIGS. 14A and 14B show a biological signal measuring probe according to a tenth embodiment of the invention. This embodiment is different from the above embodiments in shape of the probe.

In this embodiment, as shown in FIG. 14B, the light-shielding case 7 and the light guiding member 5 are concentrically disposed about the light receiving window 3 of the circular light receiving element and the light-shielding cover 6 is formed so as to expose only a part of the light-shielding case 7 and the light guiding member 5 from the bottom face of the probe. The sectional shape of the light guiding member 5 in each of the above embodiments may be applied as required.

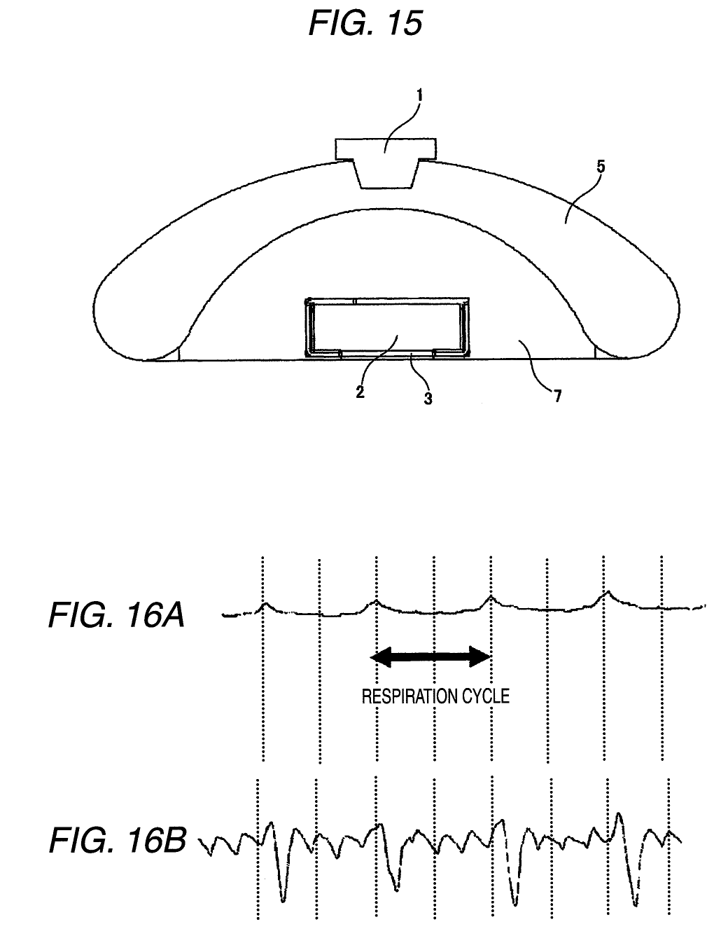

FIG. 15 shows a biological signal measuring probe according to an eleventh embodiment of the invention. In this embodiment, the LED element 1 is disposed at the vertex of the light guiding member 5. The sectional shape of upper end face of the light guiding member 5 along the optical axis of the LED element 1 is coincident with a part of the circumference of an ellipse intersecting the minor axis thereof and the optical axis of the LED element 1 is coincident with the minor axis of the ellipse, similarly to the seventh embodiment. The light emitted from the LED element 1 travels in the light guiding member 5 while being reflected by the end face of the light guiding member 5 and diffused by the light diffusing material and is emitted from the lower end face thereof to irradiate a measured object. The light reflected by the measured object enters the light receiving window 3 and is received by the light receiving element 2.

According to the biological signal measuring probe of the invention, it is possible to measure a biological signal without causing a low-temperature burn in a patient's measured portion and to enable an operator to easily mount the probe and reliably measure a biological signal with an increase in degree of freedom in selecting a patient's measured portion.

Since the light diffusing material is contained therein, the light intensity emitted from the probe is uniform regardless of the position and thus it is possible to further accurately measure a biological signal.

For example, a variation in blood amount of a peripheral vascular bed with a variation in pleural pressure due to respiration and a variation in pulse amplitude can be detected as a respiration signal from a variation in received light intensity waveform at the time of applying a single-wavelength beam to a measured object. FIGS. 16A and 16B show a received light intensity waveform when a single-waveform beam is applied with the LED element 1 directly facing a patient's skin. The interval between peaks periodically appearing in the transmitted light intensity waveform shown in FIG. 16A can be considered as a respiration cycle. FIG. 16B shows a variation in pulse amplitude (where only the pulse wave component is extracted and enlarged).

1. A probe, adapted to emit light having at least one wavelength as irradiation light with respect to a measured portion on a biological body in order to measure a biological signal, the probe comprising:
a light emitting element, configured to emit the light having the at least one wavelength;
a light guiding member, comprising:
a first end face, being light-reflective and having a first part opposing the light emitting element and a second part surrounding the first part;
a second end face, being light-reflective and intersecting the first end face; and
a third end face, being light-permeable, opposing the first end face and intersecting the second end face,
the light guiding member configured such that at least part of the light emitted from the light emitting element is reflected by at least one of the first end face and the second end face, and emitted from the third end face as the irradiation light; and
a light receiving element, configured to receive light reflected by the measured portion, wherein:
a cross-sectional shape of the first end face in a direction along an optical axis of the light emitting element is such a shape that at least the second part is coincident with a circumference of an ellipse intersecting a minor axis of the ellipse, and the optical axis of the light emitting element is coincident with the minor axis of the ellipse.

2. (canceled)

3. The probe as set forth in claim 1, wherein:
the first part of the first end face is convex with respect to the light emitting element as a first convex part; and
the second part of the first end face is concave with respect to the light emitting element.

4. The probe as set forth in claim 3, wherein:
the first convex part is semi-spherical.

5. The probe as set forth in claim 3, wherein:
the first convex part is conical.

6. The probe as set forth in claim 4, wherein:
a second convex part which is semi-spherical or conical and convex with respect to the light emitting element is formed on the first convex part.

7. The probe as set forth in claim 3, further comprising:
a support, on which the light emitting element is mounted; and
a planar mirror provided on a part of the support opposing the first end face, wherein:
the light emitted from the light emitting element is reflected by the first end face and the planar mirror to be guided to the third end face.

8. The probe as set forth in claim 3, wherein:
the light emitted from the light emitting element is reflected by the first part of the first end face and the second end face to be guided to the third end face.

9. The probe as set forth in claim 1, wherein further comprising:
a support, on which the light emitting element is mounted, wherein:
a part of the support opposing the first end face is convex with respect to the first end face and light-reflective.

10. The probe as set forth in claim 1, wherein:
the first part and the second part of the first end face are convex with respect to the light emitting element.

11. The probe as set forth in claim 1, further comprising:
a wire, electrically connected to the light emitting element and the light receiving element, wherein:
the wire is led out from the second end face.

12. The probe as set forth in claim 1, further comprising:
a board, having a first face including a first region and a second region, the board being a folded state that the first region and the second region are directed to opposite directions, wherein:
the light emitting element is disposed on the first region and the light receiving element is disposed on the second region.

13. The probe as set forth in claim 12, further comprising:
a layer disposed between the first region and the second region, and having a thermal conductivity which is lower than a thermal conductivity of the board.

14. A probe, adapted to emit light having at least one wavelength as irradiation light with respect to a measured portion on a biological body in order to measure a biological signal, the probe comprising:
a light emitting element, configured to emit the light having the at least one wavelength;
a light guiding member, comprising:
a first end face, being light-reflective and having a first part opposing the light emitting element and a second part surrounding the first part;
a second end face, being light-reflective and intersecting the first end face; and
a third end face, being light-permeable, opposing the first end face and intersecting the second end face,
the light guiding member configured such that at least part of the light emitted from the light emitting element is reflected by at least one of the first end face and the second end face, and emitted from the third end face as the irradiation light; and
a light receiving element, configured to receive light reflected by the measured portion, wherein:
a cross-sectional shape of the first end face in a direction along an optical axis of the light emitting element is such a shape that at least the second part is coincident with a circumference of an ellipse intersecting a minor axis of the ellipse;
the light emitting element is disposed at a position corresponding one of focuses of the ellipse; and
a point on the second end face is located at a position corresponding to the other one of the focuses of the ellipse.

15. The probe as set forth in claim 14, further comprising:
a wire, electrically connected to the light emitting element and the light receiving element, wherein:
the wire is led out from the second end face.

16. The probe as set forth in claim 14, further comprising:
a board, having a first face including a first region and a second region, the board being a folded state that the first region and the second region are directed to opposite directions, wherein:
the light emitting element is disposed on the first region and the light receiving element is disposed on the second region.

17. The probe as set forth in claim 16, further comprising:
a layer disposed between the first region and the second region, and having a thermal conductivity which is lower than a thermal conductivity of the board.
